(19) United States
(12) Patent Application Publication
(10) Pub. No.: US 2025/0061961 A1
(43) Pub. Date: Feb. 20, 2025

(54) MACHINE-LEARNING MODELS TO CREATE, UPDATE, VALIDATE, AND/OR ASSESS THE COMPREHENSIVENESS OF A MECHANISTIC MODEL OF BIOLOGICAL SYSTEMS

(71) Applicant: Certara, L.P., Wilmington, DE (US)

(72) Inventors: Christopher Bouton, Newbury, MA (US); Robert Allen Aspbury, Otley (GB); Pieter Hadewijin van der Graaf, Walmer (GB)

(21) Appl. No.: 18/804,983

(22) Filed: Aug. 14, 2024

Related U.S. Application Data
(60) Provisional application No. 63/533,282, filed on Aug. 17, 2023.

(51) Int. Cl. G16B 5/00; G16B 40/00

(52) U.S. Cl. CPC G16B 5/00; G16B 40/00

(57) ABSTRACT

Described herein are systems and methods for producing or editing a mechanistic model of biological systems and/or for validating (e.g., assessing the comprehensiveness of) the mechanistic model. An illustrative method produces and/or edits a mechanistic model using information derived (e.g., generated and/or identified) from a corpus comprising public and/or proprietary scientific literature using a natural language processing (NLP) machine-learning model. Producing a mechanistic model may be or include generating, modifying, extending, and/or annotating the mechanistic model. In some embodiments, a method comprises receiving, by one or more processors of one or more computing devices, a prompt. In some embodiments, the method may further include determining, by the one or more processors, information responsive to the prompt. A mechanistic model may be produced using the information, for example, by a human user.

### PRIORITY APPLICATION

This application claims the benefit of U.S. Patent Application No. 63/533,282, filed Aug. 17, 2023, which is hereby incorporated by reference herein in its entirety.

### TECHNICAL FIELD

This disclosure relates generally to artificial intelligence tools for use in producing and/or validating mechanistic models.

### BACKGROUND

Mechanistic models use first principles (e.g., laws of nature or fundamental relationships) to model physical, biological, and biochemical systems. For example, mechanistic models can be derived for biological systems and biological processes using established biological relationships, such as those between biomarkers and biological pathways. Valuable predictions, for example relevant to drug discovery, development, or testing, can then be made using such a biological mechanistic model. Mechanistic models are typically made by a person manually performing an extensive review of the state of the art regarding a particular system or process and then applying his or her expertise in the field to determine which relationships to include in the model. Mechanistic models may take the form of a knowledge graph, with nodes and associations between the nodes. Such models can take months to create, for example due to the volume of information to be previewed to create the model and/or the complexity of the system or process being modeled. There is a need, therefore, for methods and systems to produce mechanistic models in reduced time.

### SUMMARY

The present disclosure recognizes that the length of time that it takes to create mechanistic models, especially for biological systems and processes, using conventional methods presents several challenges that limit the usability of such models. For one, the models can be quite difficult to amend (e.g., extend and/or modify) after they are created and/or maintain over time as knowledge progresses. While such a limitation is not problematic initially, many areas of science, including medicine, are undergoing rapid development. Accordingly, models may become increasingly inaccurate over time with respect to current understanding of the system or process being modeled. Additionally, interest in a particular model from model users can shift over time, for example to new disease states in the pharmaceutical industry, leaving the existing model as stagnant causing it to lose value over time. As another example, while extensive effort is applied to creating mechanistic models, the universe of known information is vast and it can be easy to overlook relevant information when creating a model. For this reason, mechanistic models created solely by humans may inadvertently omit relevant information (e.g., known associations between biomarkers and biological pathways) and/or lack context (e.g., include an association that does not completely reflect current understanding). As a final example, it can be quite difficult or impossible to validate an existing mechanistic model because missing information or context would be difficult to identify.

The present disclosure provides systems and methods that incorporate machine-learning models, such as natural language processing (NLP) software and large language models (LLMs), to produce and/or validate mechanistic models, such as models of biological systems and/or biological processes. In some embodiments, a prompt is received, input into a machine-learning model, information (e.g., text) responsive to the prompt is derived (e.g., output) from (e.g., generated and/or identified with) the machine-learning model, and a mechanistic model is produced based on the information. Producing a mechanistic model may include generating a mechanistic model and/or updating a mechanistic model, such as modifying, extending and/or annotating a mechanistic model. For example, in some embodiments, a machine-learning model is used to produce an existing mechanistic model (e.g., previously generated from a human) and, in some embodiments, a machine-learning model is used to produce a new mechanistic model (i.e., that had not previously existed). In some embodiments, a mechanistic model is produced by a human user based on information determined using a machine-learning model (e.g., from a corpus of source data). For example, a human may derive mathematical formulae based on information determined using a machine-learning model that associate nodes in a model. A user may also be used to determine how a model performs in different scenarios (e.g., modelling virtual patients, use of different drug doses) in order to determine whether to further produce the mechanistic model.

By using machine-learning models (e.g., information derived therefrom) to produce a mechanistic model, the time to produce the mechanistic model may be significantly reduced (e.g., by at least 50%, at least 60%, at least 70%, at least 80%, at least 90%, or at least 95%) as compared to the time that would be required to produce an equivalent or equivalently performing mechanistic model solely using human effort and/or comprehensiveness of the model may be significantly improved (e.g., by incorporating or reflecting more information than a human would) or ensured (e.g., by validation). Moreover, such mechanistic models, or existing mechanistic models generated by humans, can be updated using methods disclosed herein, for example as scientific understanding develops.

In certain embodiments, an infrastructure is provided that includes a machine-learning model and mechanisms for deriving content from a corpus of scientific literature (e.g., public and/or proprietary) to discover evidence of associations between biological concepts and using such content to create, edit, and/or validate (e.g., assess the accuracy and/or comprehensiveness) of a biological systems mechanistic model. For example, machine learning models are used to derive parameters associated with the rate of interactions between certain biological concepts in the context of a biological systems mechanistic model. In certain embodiments, a specific corpus of private data is used as a source of information from which insights are derived (e.g., parameters associated with the rate of interactions between concepts). This may be helpful, for example, in limiting the training data that informs the machine learning model to a particular corpus that eliminates or reduces extraneous or error-prone data. In certain embodiments, the machine learning model continuously (or intermittently) updates evidence for linkages between biological concepts in a biological systems mechanistic model.

Also presented herein are methods and systems for validating, e.g., assessing the accuracy and/or comprehensiveness of a biological systems mechanistic model using a machine learning model that incorporates, for example, natural language processing (NLP) software and/or large language models (LLMs). For example, in certain embodiments, an infrastructure is provided that includes a machine learning model as well as mechanisms for deriving content from scientific literature to validate the accuracy and/or the comprehensiveness of a previously generated mechanistic model (e.g., a biological systems mechanistic model). In certain embodiments, the machine learning model may be used to derive evidence of relationships displayed in the mechanistic model, and the number of relationships derived from the machine learning model can be compared with those derived previously to generate a comprehensiveness score and/or validation score.

The mechanistic model produced, edited, and/or validated via these methods may be used, for example, in a mechanistic biosimulation platform. Such platforms include, for example, models of physiologically-based pharmacokinetics (PBPK), safety and toxicity, and quantitative systems pharmacology (QSP), all used, for example, in drug development and/or the design of clinical trials for drugs. An example of a mechanistic biosimulation platform is the Simcyp™ PBPK Simulator created by Certara, USA, which determines first-in-human dosing, optimizes clinical study design, evaluates new drug formulations, sets dose in untested populations, performs virtual bioequivalence analyses, and predicts drug-drug interactions (DDIs), for example.

In an aspect, the present invention is directed to a method of producing (e.g., generating, modifying, extending, and/or annotating) a mechanistic model using information derived (e.g., generated and/or identified) using artificial intelligence (AI), the method comprising: receiving, by one or more processors of one or more computing devices, a prompt; determining, by the one or more processors, information (e.g., text, one or more formulas, and/or numerical data) responsive to the prompt, wherein determining the information comprises inputting the prompt into a machine-learning model (e.g., a large language model (LLM)) and outputting the information from the machine-learning model (e.g., LLM); and producing (e.g., by the one or more processors) (e.g., automatically) a mechanistic model based on the information.

In some embodiments, the machine-learning model (e.g., LLM) processes the prompt using a defined corpus of source data (e.g., data fabric or data mesh) such that the information is derived (e.g., generated and/or identified) from the corpus of source data. In some embodiments, wherein the source data comprise documents (e.g., scientific literature and/or patents), data identified by IDs (e.g., PubMed® ID and/or digital object identifier (DOI)), data identified by hyperlinks (e.g., uniform resource locators (URLs)), search results (e.g., to a predetermined or user-input Internet search), or a combination thereof. In some embodiments, the source data comprise public data and the information is derived (e.g., generated and/or identified) from the public data. In some embodiments, the source data comprise proprietary (e.g., private) data and the information is derived (e.g., generated and/or identified) from the private data (e.g., and also the public data). In some embodiments, the corpus of source data has been selected based on the mechanistic model to be produced (e.g., generated, modified, extended, and/or annotated). In some embodiments, the corpus of source data has been selected based on an entity (e.g., client) (e.g., wherein the corpus of source data comprises proprietary data received from the entity). In some embodiments, the corpus of source data is fixed [e.g., comprises a pre-established set of data (e.g., regardless of the mechanistic model to be produced)]. In some embodiments, at least a portion of the corpus of source data is stored remotely from the machine-learning model (e.g., LLM) (e.g., wherein the machine-learning model (e.g., LLM) accesses the at least a portion of the corpus of source data via Internet or a local network).

In some embodiments, producing the mechanistic model comprises incorporating a source of the information in the mechanistic model (e.g., incorporating the information into the mechanistic model, the source of the information being associated with the information) such that the source of the information is determinable from the mechanistic model (e.g., by selecting an annotation in the mechanistic model). In some embodiments, the method comprises associating (e.g., by the one or more processors) a portion of the mechanistic model that has been produced with a source of the information (e.g., by associating a pointer and/or metadata with the portion of the mechanistic model). In some embodiments, the information is output from the LLM with a source of the information already associated (e.g., wherein the LLM generates a pointer and/or metadata prior to outputting the information).

In some embodiments, the mechanistic model comprises nodes [e.g., each a biological pathway, a biomarker, a biomolecule (e.g., enzyme, protein, DNA, RNA), a gene, a symptom of a disease or disorder, a disease or disorder, or a combination thereof (e.g., in a biological system and/or biological process (e.g., neurodegeneration))] and the method comprises determining, by the one or more processors, using the machine-learning model (e.g., LLM) (e.g., based on the prompt), that one or more new associations (e.g., linkages) between ones of the nodes (e.g., one or more new associations between a biomarker and a biological pathway) and/or one or more additional nodes could (e.g., should) be added to the mechanistic model. In some embodiments, the method comprises adding, by the one or more processors, the one or more new associations between ones of the nodes and/or one or more additional nodes to the mechanistic model.

In some embodiments, the information is determined by the machine-learning model (e.g., LLM) based on the mechanistic model (e.g., wherein different information would be output by the machine-learning model (e.g., LLM) for different mechanistic models).

In some embodiments, the information comprises one or more derived parameters. In some embodiments, the mechanistic model comprises nodes and the one or more derived parameters are associated with rate of interaction between nodes [e.g., each node a biological pathway, a biomarker, a biomolecule (e.g., enzyme, protein, DNA, RNA), a gene, a symptom of a disease or disorder, a disease or disorder, or a combination thereof (e.g., in a biological system and/or biological process (e.g., neurodegeneration))].

In some embodiments, the method comprises determining, by the one or more processors, using the machine-learning model (e.g., LLM), a plurality of answers to the prompt and a quality metric for each of the answers, wherein the information comprises one or more of the plurality of answers each having a respective quality metric that is higher than a respective quality of one or more other of the plurality of answers. In some embodiments, the method comprises providing, by the one or more processors, the information and the one or more other of the plurality of answers to a user in a graphical user interface (GUI) in a format (e.g., order or layout) that is based on quality (e.g., based on a quality metric determined by the machine-learning model (e.g., LLM)). In some embodiments, the method comprises providing, by the one or more processors, the information in a graphical user interface (GUI) in a format (e.g., order or layout) that is based on quality (e.g., based on a quality metric determined by the machine-learning model (e.g., LLM)) and not providing the one or more other of the plurality of answers to a user in the GUI. In some embodiments, the method further comprises receiving, by the one or more processor, a selection of the information (e.g., instead of the one or more other of the one plurality of answers) for use in producing the mechanistic model.

In some embodiments, the method comprises determining, by the one or more processors, using the machine-learning model (e.g., LLM), one or more deficiencies (e.g., missing context) in the mechanistic model. In some embodiments, producing the mechanistic model comprises modifying, extending, and/or annotating the mechanistic model based on the information such that one or more of the one or more deficiencies are mitigated or eliminated.

In some embodiments, the method comprises (e.g., automatically) continuously (e.g., every day, every week, or every month) updating the mechanistic model using the machine-learning model (e.g., LLM). In some embodiments, the method comprises updating the mechanistic model over time using subsequent output from the machine-learning model (e.g., LLM). In some embodiments, the subsequent output is derived from automatically running (e.g., periodically) preset prompts (e.g., based on the prompt and/or one or more initial prompts).

In some embodiments, the machine-learning model (e.g., LLM) outputs the information using extractive question answering (QA).

In some embodiments, the mechanistic model comprises nodes [e.g., each a biological pathway, a biomarker, a biomolecule (e.g., enzyme, protein, DNA, RNA), a gene, a symptom of a disease or disorder, a disease or disorder, or a combination thereof (e.g., in a biological system and/or biological process (e.g., neurodegeneration))] and the prompt corresponds to an association (e.g., linkage) between at least two of the nodes (e.g., an actual or potential association) (e.g., an association between a biomarker and a biological pathway). In some embodiments, the mechanistic model comprises associated nodes (e.g., after being produced) and producing the mechanistic model comprises annotating an association between at least two of the nodes [e.g., based on the information (e.g., annotating the association with the information)].

In some embodiments, the method comprises: receiving, by the one or more processors, a second prompt, wherein the second prompt is based on the mechanistic model; determining, by the one or more processors, additional information (e.g., text, one or more formulas, and/or numerical data) responsive to the second prompt, wherein determining the additional information comprises inputting the second prompt into the large language model (LLM) and outputting the additional information from the machine-learning model (e.g., LLM); and validating, by the one or more processors, the mechanistic model using the additional information.

In some embodiments, the prompt has been derived (e.g., automatically) from an existing mechanistic model (e.g., the mechanistic model before the producing). In some embodiments, the method comprises receiving (e.g., by the one or more processors) a selection (e.g., made with user input into a graphical user interface (GUI)) of an association in the mechanistic model; and determining the prompt (e.g., automatically) (e.g., by the one or more processors) based on the selection.

In some embodiments, determining the information comprises generating at least a portion of the information. In some embodiments, determining the information comprises identifying at least a portion of the information in a corpus of source data.

In some embodiments, the machine-learning model (e.g., LLM) is a base model. In some embodiments, the machine-learning model (e.g., LLM) is a fine-tuned model. In some embodiments, the machine-learning model (e.g., LLM) is a commercially available model. In some embodiments, the machine-learning model (e.g., LLM) is a purpose-built model. In some embodiments, the machine-learning model (e.g., LLM) is a generative pre-trained transformer (GPT). In some embodiments, the machine-learning model (e.g., LLM) is a foundation model.

In some embodiments, the method comprises storing the produced (e.g., generated, modified, extended, and/or annotated) mechanistic model in one or more non-transitory computer readable media (e.g., as a knowledge graph).

In some embodiments, the mechanistic model comprises (e.g., is) a quantitative systems pharmacology (QSP) model or physiologically based pharmacokinetics (PBPK) model. In some embodiments, the mechanistic model is a model for a biological system and/or a biological process (e.g., neurodegeneration).

In some embodiments, the information is related to a biological pathway, a biomarker, a biomolecule (e.g., enzyme, protein, DNA, RNA), a gene, a symptom of a disease or disorder, a disease or disorder, or a combination thereof. In some embodiments, the prompt is based on (e.g., corresponds to an actual or potential association between) a biological pathway of interest, a biomarker of interest, a biomolecule (e.g., enzyme, protein, DNA, RNA) of interest, a gene of interest, a symptom of a disease or disorder of interest, a disease or disorder of interest, or a combination thereof.

In some embodiments, the information has been processed (e.g., summarized) from source data used by the machine-learning model (e.g., LLM) (e.g., wherein the output from the machine-learning model (e.g., LLM) is a summarized version of information contained in source data used by the machine-learning model (e.g., LLM)). In some embodiments, the method comprises processing the information, wherein producing the mechanistic model based on the information occurs based on the processed information.

In some embodiments, the prompt is received via a plugin (e.g., to a text editor), web app, or application programming interface (API) (e.g., wherein the machine-learning model (e.g., LLM) is accessible via the plugin, the web app, or the API, respectively).

In some embodiments, the prompt is a user-input prompt. In some embodiments, the prompt is an automatically provided prompt. In some embodiments, the prompt is a template prompt or a prompt derived from a template prompt.

In some embodiments, the method comprises determining or predicting a dosing regimen (e.g., an improved dosing regimen) based on the mechanistic model. In some embodiments, the method comprises determining or predicting suitability (e.g., effectiveness and/or safety) of a pharmaceutical composition [e.g., comprising a drug (e.g., a biologic)] (e.g., at treating a disorder or disease) based on the mechanistic model.

In another aspect, the present invention is directed to a method of validating a mechanistic model, the method comprising: receiving, by one or more processors of one or more computing devices, a mechanistic model; inputting, by the one or more processors, at least a portion of the mechanistic model into a machine-learning model (e.g., a large language model (LLM)); and determining, by the one or more processors, a comprehensiveness of the mechanistic model based on output from the machine-learning model (e.g., wherein the output is the comprehensiveness). In some embodiments, the mechanistic model had been generated solely by one or more humans. In some embodiments, the mechanistic model is a human-generated model.

In some embodiments, determining the comprehensiveness comprises determining, by the machine-learning model, a comprehensiveness score (e.g., metric) (e.g., similarity index) for the mechanistic model (e.g., for the at least a portion of the mechanistic model). In some embodiments, determining the comprehensiveness score comprises: receiving, by the one or more processors, a prompt; determining, by the one or more processors, information (e.g., text, one or more formulas, and/or numerical data) responsive to the prompt, wherein determining the information comprises inputting the prompt into the machine-learning model and outputting the information from the machine-learning model; and comparing, by the one or more processors, the information to the mechanistic model. In some embodiments, the method comprises determining, by the one or more processors, the prompt based on the mechanistic model (e.g., automatically determined by the machine-learning model). In some embodiments, the mechanistic model comprises nodes and associations between ones of the nodes and determining the comprehensiveness score comprises determining, by the one or more processors, whether one or more associations and/or one or more nodes could (e.g., should) be added to the mechanistic model.

In some embodiments, determining the comprehensiveness comprises: receiving, by the one or more processors, a prompt; determining, by the one or more processors, information (e.g., text, one or more formulas, and/or numerical data) responsive to the prompt, wherein determining the information comprises inputting the prompt into the machine-learning model and outputting the information from the machine-learning model; and comparing, by the one or more processors, the information to the mechanistic model.

In some embodiments, the method comprises updating (e.g., extending and/or modifying), by the one or more processors, the mechanistic model using the machine-learning model based on the comprehensiveness. In some embodiments, the method comprises determining, by the one or more processors, that a comprehensiveness score for the mechanistic model is below a threshold (e.g., that a comprehensiveness score of a portion of the mechanistic model is below a threshold for that portion of the mechanistic model), wherein the updating occurs subsequently.

In some embodiments, the mechanistic model comprises nodes and associations between ones of the nodes and the at least a portion of the mechanistic model comprises at least one of the nodes and/or at least one of the associations.

In some embodiments, the machine-learning model uses the at least a portion of the mechanistic model and a defined corpus of source data (e.g., data fabric or data mesh) to derive (e.g., generate and/or identify) the output. In some embodiments, the source data comprise documents [e.g., scientific literature and/or patents], data identified by IDs (e.g., PubMed® ID and/or digital object identifier (DOI)), data identified by hyperlinks (e.g., uniform resource locators (URLs)), search results (e.g., to a predetermined or user-input Internet search), or a combination thereof. In some embodiments, the source data comprise public data and the output is derived (e.g., generated and/or identified) from the public data. In some embodiments, the source data comprise proprietary (e.g., private) data and the output is derived (e.g., generated and/or identified) from the private data (e.g., and also the public data). In some embodiments, the corpus of source data has been selected based on the mechanistic model. In some embodiments, the corpus of source data has been selected based on an entity (e.g., client) corresponding to the mechanistic model (e.g., wherein the corpus of source data comprises proprietary data received from the entity). In some embodiments, the corpus of source data is fixed [e.g., comprises a pre-established set of data (e.g., regardless of the mechanistic model)]. In some embodiments, at least a portion of the corpus of source data is stored remotely from the machine-learning algorithm (e.g., wherein the machine-learning algorithm accesses the at least a portion of the corpus of source data via Internet or a local network).

In some embodiments, the machine-learning model is a base model. In some embodiments, the machine-learning model is a fine-tuned model. In some embodiments, the machine-learning model is commercially available model. In some embodiments, the machine-learning model is a purpose-built model. In some embodiments, the machine-learning model is a generative pre-trained transformer (GPT). In some embodiments, the machine-learning model is a foundation model.

In another aspect, the present invention is directed to a system comprising one or more processors and one or more non-transitory computer readable media, wherein the one or more non-transitory computer readable media have instructions stored thereon that, when executed by the one or more processors, cause the one or more processors to perform operations comprising a method disclosed herein.

In another aspect, the present invention is directed to one or more non-transitory computer readable media having instructions stored thereon that, when executed by one or more processors, cause the one or more processors to perform operations comprising a method disclosed herein.

Any two or more of the features described in this specification, including in this summary section, may be combined to form implementations of the disclosure, whether specifically expressly described as a separate combination in this specification or not.

At least part of the methods, systems, and techniques described in this specification may be controlled by executing, on one or more processing devices, instructions that are stored on one or more non-transitory machine-readable storage media. Examples of non-transitory machine-readable storage media include read-only memory, an optical disk drive, memory disk drive, and random access memory. At least part of the methods, systems, and techniques described in this specification may be controlled using a computing system comprised of one or more processing devices and memory storing instructions that are executable by the one or more processing devices to perform various control operations.

### Definitions

In order for the present disclosure to be more readily understood, certain terms used herein are defined below. Additional definitions for the following terms and other terms may be set forth throughout the specification.

In this application, unless otherwise clear from context or otherwise explicitly stated, (i) the term “a” may be understood to mean “at least one”; (ii) the term “or” may be understood to mean “and/or”; and (iii) the terms “comprising” and “including” may be understood to encompass itemized components or steps whether presented by themselves or together with one or more additional components or steps.

Pharmaceutical composition: As used herein, the term “pharmaceutical composition” refers to a composition in which an active agent is formulated together with one or more pharmaceutically acceptable carriers. In some embodiments, the active agent is present in unit dose amount appropriate for administration in a therapeutic regimen that shows a statistically significant probability of achieving a predetermined therapeutic effect when administered to a relevant population. In some embodiments, a pharmaceutical composition may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces. A pharmaceutical composition may comprise a small molecule and/or biologic.

Treatment: As used herein, the term “treatment” (also “treat” or “treating”) refers to any administration of a therapy (e.g., pharmaceutical composition) that partially or completely alleviates, ameliorates, relives, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

Dosing regimen: Those skilled in the art will appreciate that the term “dosing regimen” may be used to refer to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount In some embodiments, a dosing regimen is correlated with a desired or beneficial outcome when administered across a relevant population (i.e., is a therapeutic dosing regimen).

Effectiveness: As used herein, means that a pharmaceutical composition, when administered in a sufficient amount, produces the desired effect for which it is administered. In some embodiments, the term refers to an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, one or more symptoms of the disease, disorder, and/or condition.

Machine-learning model: As used herein, the term “machine-learning model” is used to refer to a computer implemented process (e.g., a software function) that implements one or more particular machine learning algorithms, such as natural language processing models (NLPs), large language models (LLMs), generative pre-trained transformers (GPTs), artificial neural networks (ANN), convolutional neural networks (CNNs), random forest, decision trees, support vector machines, and the like, in order to determine, for a given input, one or more output values. A machine-learning model may use a transformer architecture, such as in a GPT. A machine-learning model may by a large language model. In some embodiments, machine learning models implementing machine learning techniques are trained, for example using curated and/or manually annotated datasets. Such training may be used to determine various parameters of machine learning algorithms implemented by a machine-learning model, such as weights associated with layers in neural networks. In some embodiments, once a machine learning model is trained, e.g., to accomplish a specific task such as generating and/or identifying information as described herein, values of determined parameters are fixed and the (e.g., unchanging, static) machine-learning model is used to process new data (e.g., different from the training data) and accomplish its trained task without further updates to its parameters (e.g., the machine-learning model does not receive feedback and/or updates). In some embodiments, a machine-learning model may receive feedback, e.g., based on user review of accuracy, and such feedback may be used as additional training data, for example to dynamically update the machine-learning model. In some embodiments, two or more machine learning models may be combined and implemented as a single complex model and/or a single software application. In some embodiments, two or more machine-learning models may also be implemented separately, e.g., as separate software applications. A machine-learning model may be software and/or hardware. For example, a machine learning module may be implemented entirely as software, or certain functions of a ANN module may be carried out via specialized hardware (e.g., via an application specific integrated circuit (ASIC), field programmable gate arrays (FPGAs), and the like). In certain embodiments, this NLP software makes use of one or more large language models (LLMs). In certain embodiments, the one or more LLMs may be partially or wholly proprietary. In certain embodiments, the one or more LLMs may include one or more of the following known LLMs: BERT (Google) (or other transformer-based models), Falcon 40B, Galactica, GPT-3 (Generative Pre-trained Transformer, OpenAI), GPT-3.5 (OpenAI), GPT-4 (OpenAI), LaMDA (language model for dialogue applications, Google), Llama (large language model Meta AI) (Meta), Orca LLM (Microsoft), PaLM (Pathways Language Model), Phi-1 (Microsoft), StableLM (Stability AI), BLOOM (Hugging Face), ROBERTa (Meta), XLM-ROBERTa (Meta), NeMO LLM (Nvidia), XLNet (Google), Generate (Cohere), GLM-130B (Hugging Face), Claude (Anthropic). The one or more LLMs may include one or more autoregressive LLMs, autoencoding LLMs, encoder-decoder LLMs, bidirectional LLMs, fine-tuned LLMs, and/or multimodal LLMs.

### BRIEF DESCRIPTION OF THE DRAWINGS

The present teachings described herein will be more fully understood from the following description of various illustrative embodiments, when read together with the accompanying drawings. It should be understood that the drawing described below is for illustration purposes only and is not intended to limit the scope of the present teachings in any way. The foregoing and other objects, aspects, features, and advantages of the disclosure will become more apparent and may be better understood by referring to the following description taken in conjunction with the accompanying drawings, in which:

FIG. 1 illustrates a system for producing and/or validating mechanistic models using machine-learning models, according to illustrative embodiments of the present disclosure;

FIG. 2 is a flow diagram of a method, according to illustrative embodiments of the present disclosure;

FIG. 3 is a flow diagram of a method, according to illustrative embodiments of the present disclosure;

FIG. 4 is an illustration of graphical user interfaces for producing mechanistic models for biological systems or processes using a large language model, according to illustrative embodiments of the present disclosure;

FIG. 5 is an illustration of a graphical user interface for using a large language model, according to illustrative embodiments of the present disclosure;

FIG. 6 is an illustration of a graphical user interface for using a large language model, according to illustrative embodiments of the present disclosure;

FIG. 7 is a schematic showing an illustrative implementation of a network environment for use in providing systems, methods, and architectures as described herein; and

FIG. 8 is a schematic showing an illustrative network environment for use in providing systems, methods, and architectures as described herein.

### DETAILED DESCRIPTION OF CERTAIN EMBODIMENTS

Described herein are systems and methods for producing mechanistic models and for validating mechanistic models. In some embodiments, a method produces a mechanistic model using information derived (e.g., generated and/or identified) using artificial intelligence (AI), such as a machine-learning model. Producing a mechanistic model may be or include generating, modifying, extending, and/or annotating the mechanistic model. For example, in some embodiments, a machine-learning model is used to produce an existing mechanistic model by modifying, extending, and/or annotating the model. In some embodiments, a machine-learning model is used to produce a mechanistic model by generating the model. In some embodiments, a machine-learning model is used to generate a mechanistic model and then subsequently used again to produce the mechanistic model by extending, modifying, and/or annotating the model. Mechanistic models may be produced automatically, for example a machine-learning model may produce a mechanistic model such that output from the machine-learning model includes the mechanistic model.

### Producing Mechanistic Models

In some embodiments, a machine-learning model is used to determine (e.g., generate and/or identify) information and a mechanistic model is produced based on the information (e.g., is produced to include the information). Information determined by a machine-learning model may be, for example, e.g., text, one or more formulas, and/or numerical data. In some embodiments, a method comprises receiving, by one or more processors of one or more computing devices, a prompt. In some embodiments, the method may further include determining, by the one or more processors, information responsive to the prompt. In some embodiments, determining the information comprises inputting the prompt into a machine-learning model (e.g., an LLM) and outputting the information from the machine-learning model. A mechanistic model may be producing (e.g., by the one or more processors) (e.g., automatically) based on the information. Determining the information may comprise generating at least a portion of the information. Determining the information may comprise identifying at least a portion of the information in a corpus of source data. In some embodiments, a mechanistic model comprises associated nodes (e.g., after being produced) and producing a mechanistic model comprises annotating an association between at least two of the nodes [e.g., based on information (e.g., annotating the association with the information)]. As discussed further subsequently, a mechanistic model may be alternatively or additionally validated using a machine-learning model (e.g., the same machine learning model used to produce the mechanistic model). In some embodiments, a mechanistic model may be validated at a time subsequent (e.g., in days, weeks, or months) to when a mechanistic model is produced.

A mechanistic model produced using a method disclosed herein may be used to determine or predict a dosing regimen based on the mechanistic model. The dosing regimen may be improved relative to a conventional mechanistic model, for example because the comprehensiveness of the mechanistic model is greater. Additionally or alternatively, a mechanistic model produced using a method disclosed herein may be used to determine or predict suitability (e.g., effectiveness and/or safety) of a pharmaceutical composition [e.g., comprising a drug (e.g., a biologic)] (e.g., at treating a disorder or disease) based on the mechanistic model.

In some embodiments, information is determined (e.g., generated and/or identified) by a machine-learning model based on a mechanistic model to be produced. A machine-learning model may consider information about a mechanistic model in. Thus, in some embodiment, different information would be output by a machine-learning model for different mechanistic models. In some embodiments, information determined (e.g., generated and/or identified) from a machine-learning model comprises one or more derived parameters. In some embodiments, the mechanistic model comprises nodes and the one or more derived parameters are associated with rate of interaction between nodes, such as, for example a rate of interaction between a biological pathway and a biomarker.

A machine-learning model may generate and/or identify a significant amount of information responsive to a prompt. For example, a machine-learning model may generate and/or identify a large section of text from and/or in a single source (e.g., document) and/or may identify many sources of information within a corpus of source data. In order to provide useful information to a user, a machine-learning model may determine a quality metric for different portions of information. Information may be presented to a user in a GUI based on the quality metric. In some embodiments, a method comprises determining a plurality of answers to a prompt for a machine-learning model and a quality metric for each of the answers. Information output by the machine-learning model may comprise one or more of the plurality of answers each having a respective quality metric that is higher than a respective quality of one or more other of the plurality of answers. In some embodiments, a method comprises providing information to a user in a GUI in a format (e.g., order or layout) that is based on quality (e.g., based on a quality metric determined by a machine-learning model). In some embodiments, a method comprises providing to a user some information responsive to a prompt to a machine-learning model and not providing other information also responsive to the prompt to the user in a GUI (e.g., based on a quality metric determined by the LLM). The information that is provided may be provided in a format (e.g., order or layout) that is based on quality. A user may select information presented in a GUI (e.g., using one or more widgets in the GUI) for use in producing the mechanistic model. Thus, not all information generated and/or identified by a machine-learning model is necessarily used to produce a mechanistic model. A user may still control (e.g., by applying his or her expertise) production of a mechanistic model.

Use of machine-learning models to produce and/or validate mechanistic models as disclosed herein enables mechanistic models to be produced [e.g., updated (e.g., extended and/or modified)] over time, which is highly impractical, if not impossible, to do for human created (and maintained) mechanistic models. For example, in some cases, even where a human could possibly update a mechanistic model, it may be so laborious to update the mechanistic model and new relevant data may be generated or otherwise become available so quickly that the model would be outdated again by the time that it was finished being updated. Use of machine-learning models as disclosed herein not only enables updating mechanistic models in practically feasible time frames but also the ability to automatically and/or continuously produce (e.g., update) mechanistic models. In some embodiments, a method comprises (e.g., automatically) continuously (e.g., every day, every week, or every month) updating a mechanistic model using machine-learning model. In some embodiments, a method comprises updating a mechanistic model over time using subsequent output from a machine-learning model. In some embodiments, subsequent output is derived from automatically running (e.g., periodically) preset prompts (e.g., based on the prompt and/or one or more initial prompts).

A mechanistic model may have or be produced to have nodes and associations (e.g., linkages) between ones of the nodes. The nodes may each correspond to a biological pathway, a biomarker, a biomolecule (e.g., enzyme, protein, DNA, RNA), a gene, a symptom of a disease or disorder, or a disease or disorder. The associations (e.g., linkages) may correspond to relationships (e.g., known in the literature or inherently included in a corpus of source data) between ones of the nodes, for example a relationship between a biomarker and a biological pathway. Prompts to a machine-learning model may be based on nodes and/or associations that are or may be included in a mechanistic model. A machine-learning model may be used to determine that one or more new associations (e.g., linkages) between ones of the nodes (e.g., one or more new associations between a biomarker and a biological pathway) and/or one or more additional nodes could (e.g., should) be added to the mechanistic model. The one or more new associations between ones of the nodes and/or one or more additional nodes may be then added to the mechanistic model.

In some embodiments, a mechanistic model comprises or is a quantitative systems pharmacology (QSP) model or physiologically based pharmacokinetics (PBPK) model. In some embodiments, a mechanistic model is a model for a biological system and/or a biological process (e.g., neurodegeneration). In some embodiments, information derived from a machine-learning model is related to a biological pathway, a biomarker, a biomolecule (e.g., enzyme, protein, DNA, RNA), a gene, a symptom of a disease or disorder, a disease or disorder, or a combination thereof.

In some embodiments, output (e.g., information) derived from a machine-learning model may be processed and/or have been processed from source data. Output (e.g., information) may be processed to provide context, tabulate (e.g., numerical data), and/or summarize, for example. For example, a machine-learning model may be used to identify information (e.g., text) in a corpus of source data that is responsive to a prompt and the identified text may be processed to be summarized, either prior to being output by the machine-learning model or after being output. Producing a mechanistic model based on information may occur based on the information after processing it. Information output from a machine-learning model may have been processed (e.g., summarized) from source data used by the machine-learning model (e.g., wherein the output from the machine-learning model is a summarized version of information contained in source data used by the LLM).

### Mechanistic Model Validation

Disclosed herein are methods for validating mechanistic models by using machine-learning models, for example LLMs. Absent methods disclosed herein, a mechanistic model can be difficult or impossible to validate given the length of time and complexity involved in making such models. Validated mechanistic models may also ensure determinations made from or other uses of mechanistic models can be trusted to be accurate, which may be important for satisfying requirements of regulatory agencies (e.g., the United States Food and Drug Administration). In some embodiments, a method comprises receiving, by one or more processors of one or more computing devices, a mechanistic model. The mechanistic model may be in the form of a knowledge graph. The mechanistic model may include nodes and associations between the nodes. At least a portion of the mechanistic model may be input by the one or more processors into a machine-learning model (e.g., a LLM). For example, one or more nodes and/or one or more associations (e.g., a subgraph of a knowledge graph) may be input into a machine-learning model. A comprehensiveness of the mechanistic model may be determined based on output from the machine-learning model. In some embodiments, comprehensiveness of a mechanistic model is output from a machine-learning model. For example, a machine-learning model may compare nodes and/or associations in a mechanistic model with information in a defined corpus of source data to determine whether one or more additional nodes and/or one or more additional associations are missing from the mechanistic model and/or whether one or more associations present in the mechanistic model lack context and output a representation of comprehensiveness based on the results of the comparison. Comprehensiveness may be represented using any suitable scale, for example binary (comprehensive or not comprehensive), qualitative (e.g., very comprehensive, moderately comprehensive, somewhat comprehensive, or not comprehensive), or quantitative (e.g., a comprehensiveness score, such as on a scale of 1-100). In some embodiments, comprehensiveness of a mechanistic model that had been generated solely by one or more humans may be determined. In some embodiments, comprehensiveness of a human-generated mechanistic model may be determined.

In some embodiments, determining comprehensiveness comprises determining, (e.g., by a machine-learning model) a comprehensiveness score (e.g., metric) (e.g., similarity index) for the mechanistic model. Comprehensiveness of a mechanistic model may be determined based on a comprehensiveness score for at least a portion of (e.g., only a portion of) a mechanistic model, for example a portion of interest, such as a portion relevant to a new scientific breakthrough. In some embodiments, determining a comprehensiveness score comprises receiving, by one or more processors, a prompt; determining, by the one or more processors, information (e.g., text, one or more formulas, and/or numerical data) responsive to the prompt; and comparing, by the one or more processors, the information to the mechanistic model. In some embodiments, determining the information comprises inputting the prompt into the machine-learning model and outputting the information from the machine-learning model. In some embodiments, the prompt may be determined based on the mechanistic model (e.g., automatically determined by the machine-learning model). In some embodiments, a mechanistic model comprises nodes and associations between ones of the nodes and a comprehensiveness score may be determined at least by determining whether one or more associations and/or one or more nodes could (e.g., should) be added to the mechanistic model. In some embodiments, determining comprehensiveness of a mechanistic model comprises: receiving, by one or more processors, a prompt; determining, by the one or more processors, information (e.g., text, one or more formulas, and/or numerical data) responsive to the prompt; and comparing, by the one or more processors, the information to the mechanistic model. In some embodiments, determining the information comprises inputting the prompt into a machine-learning model and outputting the information from the machine-learning model.

In some embodiments, a machine-learning model used to determine comprehensiveness of a mechanistic model may also be used to update (e.g., by one or more processors) the mechanistic model. In some embodiments, a method comprises determining (e.g., by one or more processors) that a comprehensiveness score for a mechanistic model is below a threshold. For example, a comprehensiveness score of a portion of the mechanistic model may be determined to be below a threshold for that portion of the mechanistic model. The updating may occur subsequently.

In some embodiments, a method (e.g., of producing a mechanistic model) comprises determining, by the one or more processors, using a machine-learning model, one or more deficiencies (e.g., missing context) in a mechanistic model. In some embodiments, a method comprises modifying, extending, and/or annotating the mechanistic model based on information determined by a machine-learning model responsive to a prompt such that one or more one or more deficiencies are mitigated or eliminated.

In some embodiments, a method of producing a mechanistic model comprises receiving a second prompt. The second prompt may be based on the mechanistic model. In some embodiments, a method comprises determining additional information (e.g., text, one or more formulas, and/or numerical data) responsive to the second prompt. Determining the additional information may comprise inputting the second prompt into a machine-learning model (e.g., used to produce a mechanistic model) and outputting the additional information from the machine-learning model. Finally, the mechanistic model may be validated (e.g., by the one or more processors) using the additional information.

### Source Data for Use in Producing Mechanistic Models

Methods disclosed herein produce improved mechanistic models in that an association between a source of information and the information itself can be made and easily integrated into the mechanistic model. Commercially available machine-learning models, such as GPT-4 or LLAMA 2, by themselves can generate answers to prompts that may be accurate and even quite complex. However, such models are known to hallucinate information. There is no way to know whether the information generated by a model is actually accurate without independent verification. Independent verification may be difficult because the source of the information is not known. While such models could be asked to provide a source, the models can also hallucinate the sources themselves such that an accurate original source of information may never be known. In contrast, disclosed herein are methods that use machine-learning models (e.g., LLMs) that use a defined corpus of source data to determine (e.g., generate and/or identify) information responsive to a prompt. Because the information is determined based on a defined corpus of source data, the source (e.g., one or more documents) of the information can be associated with the information and the source of the information is known to be accurate. A machine-learning model may use extractive question answer (QA) to determine information responsive to a prompt, which may reduce the likelihood of hallucinated information as compared to, for example, a generative approach even when such generative approach uses a defined corpus of source data. A defined corpus of source data may comprise discrete and/or traceable sources of information, such as documents [e.g., scientific literature and/or patents], data identified by IDs (e.g., PubMed® ID and/or digital object identifier (DOI)), data identified by hyperlinks (e.g., uniform resource locators (URLs)), search results (e.g., to a predetermined or user-input Internet search), or a combination thereof.

In some embodiments, a method comprises associating a portion of a mechanistic model that has been produced based on information with a source of the information. For example, information and its source may be associated by associating a pointer and/or metadata with the information. Producing a mechanistic model may include incorporating the information and associating a source of the information with the information that is incorporated. In some embodiments, information is output from a machine-learning model with the source of the information already associated. For example, a machine-learning model may generate a pointer and/or a metadata prior to outputting the information. In some embodiments, producing a mechanistic model comprises incorporating a source of the information into the mechanistic model (e.g., incorporating the information into the mechanistic model, the source of the information being associated with the information) (e.g., as an annotation) such that the source of the information is determinable from the mechanistic model. For example, a source of information may be determinable by selecting an annotation in the mechanistic model.

In some embodiments, a machine-learning model (e.g., LLM) processes a prompt using a defined corpus of source data (e.g., a data fabric or data mesh) such that information is derived (e.g., generated and/or identified) from the corpus of source data. In some embodiments, source data in a corpus comprise documents, data identified by IDs, data identified by hyperlinks (e.g., uniform resource locators (URLs)), search results, or a combination thereof. Documents may be from scientific literature and/or may be patents. Scientific literature may be from open-access journals (e.g., arXiv or bioRxiv) and/or closed-access (pay to access) journals. Data identified by IDs may be identified by, for example, a PubMed® ID and/or a digital object identifier (DOI). Data from search results may be data responsive to a predetermined or user-input Internet search, for example on a publicly available search engine. In some embodiments, source data comprise public data. Information may be derived (e.g., generated and/or identified) from the public data using a machine-learning model. In some embodiments, source data comprise proprietary (e.g., private) data. Information may be derived (e.g., generated and/or identified) from the proprietary data (e.g., and also from public data) using a machine-learning model. In some embodiments, at least a portion of a corpus of source data is stored remotely from a machine-learning model. For example, a machine-learning model may access the at least a portion of the corpus of source data via the Internet or a local network. Systems and methods for using machine-learning models with a corpus of source data are disclosed in U.S. Pat. No. 11,216,752, the disclosure of which is incorporated by reference herein in its entirety.

In some embodiments, a corpus of source data has been selected based on a mechanistic model to be produced (e.g., generated, modified, extended, and/or annotated). In some embodiments, a corpus of source data has been selected based on an entity (e.g., client). For example, a corpus of source data used by a machine-learning model may comprise proprietary data received from the entity. In some embodiments, a corpus of source data is fixed. For example a corpus of source data may be a pre-established set of data, for example that does not change regardless of the mechanistic model to be produced.

In some embodiments, a prompt is provided to a machine-learning model as input, for example in order to generate information based on a corpus of source data and/or identify information in a corpus of source data. A prompt may be a user-input prompt, for example input with a GUI. A prompt may be an automatically provided prompt, for example provided automatically to a machine-learning model as input on a continuous basis. A prompt may be a template prompt or a prompt derived from a template prompt. For example, a template prompt may be established (e.g., by a user) based on a particular mechanistic model or a particular type of mechanistic model to be produced. In some embodiments, a prompt has been derived from an existing mechanistic model. The existing mechanistic model may be produced (e.g., modified and/or extended) based on output from a machine-learning model using the prompt. A prompt may be automatically derived using a machine-learning model. In some embodiments, a selection (e.g., made with user input into a graphical user interface (GUI)) of an association in a mechanistic model is made and a prompt (e.g., automatically) based on the selection. For example, a template prompt or prompt derived from a template prompt may be automatically determined based on an association (e.g., linkage) in a mechanistic model that is selected (e.g., by a user) (e.g., automatically, for example if checking all associations in a mechanistic model as part of a continuous update).

### Machine-Learning Models

In some embodiments, one or more steps of a method disclosed herein are performed using a machine-learning model. The machine-learning model may be a large language model, such as a generative pre-trained. The machine-learning model may be a generative pre-trained transformer. The machine-learning model may be trained to perform question-answer (QA) extraction. The machine-learning model may be trained to generate and/or identify text, for example from a corpus of source data. The machine-learning model may be a base model. The machine-learning model may be a fine-tuned model, for example using supervised or reinforcement learning. A fine-tuned model may be fine-tuned using proprietary source data, for example in order to improve quality of output from the machine-learning model for a particular use related to the proprietary source data, such as producing a relevant mechanistic model. As an example, proprietary experimental data (e.g., clinical data) may be used to fine-tune a machine-learning model for the purpose of producing and/or validating mechanistic models for biological systems and/or processes related to the experimental data. Fine-tuning a machine-learning model may reduce hallucinations if generating text. The machine-learning model may be an open-sourced model. The machine-learning model may be a proprietary model. A proprietary model may be a commercially available model, such as, for example, GPT-4 or LLAMA 2, or a purpose-built model [e.g., trained exclusively using a desired (e.g., predetermined) corpus of source data]. A purpose-built model may be less likely to hallucinate text, may benefit from using a more up-to-date corpus of source data, and/or be more secure (e.g., by dint of remaining behind a critical safety firewall). The machine-learning model may be a foundation model.

In some embodiments, a machine-learning model is accessed via a plugin (e.g., to a text editor), web app, or application programming interface (API). In some embodiments a prompt is received via a plugin (e.g., to a text editor), web app, or application programming interface (API). In some embodiments, a mechanistic model that has been produced (e.g., generated, modified, extended, and/or annotated) may be stored in one or more non-transitory computer readable media (e.g., as a knowledge graph).

### Mechanistic Models

Mechanistic models can be used to model a variety of systems and processes. One particularly important, and common, example field where mechanistic models are used is biological systems and processes. Mechanistic models have been used in drug development and to model clinical trials. For example, a mechanistic model may be made for a particular disease state in order to model how a drug affects the disease state. In another example, a mechanistic model may be used to simulate a drug's effect on a simulated patient population in order to predict clinical trial outcome.

Mechanistic models can use mathematical relationships as associations between nodes in a model in order to make predictions based on first principles. For example, a mechanistic model may incorporate mathematical relationships of pathophysiologie processes at varying degrees of granularity associated with clinical disease phenotypes. As another example, nodes corresponding to genes and/or proteins may be mathematically associated in a mechanistic model that models cellular behavior in order to model impact of pathologic or therapeutic perturbations on cellular operation and therefore clinical phenotype.

Mechanistic models have advantages that derive from their basis in first principles, as compared to statistical or empirical models. Determined parameters have actual physical meaning, which facilitates further interpretation of results. Additionally, the output of a model is often valid beyond the calibration space and in broad range of scenarios where other parameters are changing.

Model generation often starts with a design phase where literature that is related to the problem at hand is analyzed and specific associations between individual model nodes are derived. As a model becomes more complex, the amount of literature that one has to analyze grows with it and its comprehensive analysis becomes more and more labor-intensive. The next phase, architecture, is concerned with building a general structure of the model, developing governing equations, and defining parameters. In most modern cases, model implementation occurs in a programming environment for subsequent execution and internal validation that, in turn, identifies model constraints and calibrates the model. The task of implementation scales with model complexity. External validation can be performed where results are verified with additional perturbations. In some embodiments, the model can be further revised to adapt for new targets and to incorporate updates as new data emerges, though doing so may be difficult depending on the nature of the model and the nature of the development in scientific understanding. For example, such revision can require analysis and monitoring of a large amount of literature.

A specific use area of mechanistic models is pharmacology where a model can help to optimize therapeutic effects, such as drug dosing and its benefits. In particular, many mathematical models have been developed recently to aid studying the mechanisms of drug pharmacokinetics, pharmacodynamics, and disease processes, often referred to as mechanistic biosimulation platforms. Two examples of mechanistic biosimulation platforms are physiologically based pharmacokinetic (PBPK) platforms and quantitative systems pharmacology (QSP) platforms. PBPK focuses on making pharmacokinetic predictions (e.g., how a system affects a drug), whereas QSP is concerned with pharmacodynamics (e.g., how the drug affects the system) and clinical efficacy outcomes. Taken together, pharmacokinetics and pharmacodynamics influence dosing, benefits, and adverse effects of drugs.

A typical setup of a biosimulation platform that implements both QSP and PBPK involves representing a living system as plurality of compartments by grouping organs or tissues. Groupings can be made based on, for example, similarity of blood perfusion rate and/or lipid content. A model can define ports of entry for drugs (e.g., skin, lung, and intestinal tract), ports of exit (e.g., liver, kidney), and target organs for studying therapeutic effect or toxicity. The interaction between compartments and a drug is described using a variety of pharmacokinetic variables, such as in an absorption, distribution, metabolism, and excretion (ADME) framework or its variations. Connections between compartments follow physiology. A model can be defined by sets of ordinary differential equations that follow the principles of fluid dynamics, mass transport, and biochemistry, simulating the fate of a drug in the system.

Optimal drug dosing and benefits have invaluable importance at all stages of drug development. By their design, biosimulation platforms provide the ability to describe mathematically any changes in one's physiology or drug properties and, in turn, predict therapeutic outcomes in a given scenario. These platforms, therefore, allow studying effects of these changes in silico prior to the actual dosing, providing direct health, time, labor, and cost savings. For example, when translating clinical trial outcomes to the real-world population, a biosimulation platform may evaluate effects of previously unrecognized or understudies physiological parameters that directly impact drug pharmacokinetics. The parameters may include various extrinsic (e.g., pregnancy, genetics, disease, organ dysfunction, race) and intrinsic factors (e.g., diet, smoking, alcohol use, other medications). In another aspect, a model can capture various drug formulations and determine in silico their therapeutic effects, shortening and narrowing drug development stages. Of particular interest is the ability to translate the success of clinical trials between different species. For instance, in scenarios where initial trials were performed in non-human system, a biosimulation platform may evaluate the performance of the drug in human, predict first in-human dosing, and optimize pharmacokinetic properties for the highest benefit. Such predictions may reduce or eliminate the need for human clinical trials or may optimize clinical trial design to increase likelihood of success, shorten trial time, or reduce the number of trials to determine safety and efficacy.

One example of a biosimulation platform is Simcyp™ PBPK Simulator (Simcyp) created by Certara. Among other things, Simcyp provides an interface for a user to input mechanistic details of a model in the form of diagrams and biological maps. The interface allows to capture expert opinions, to quickly implement recent literature findings, and to facilitate collaboration between scientists with wide range of technical expertise and scientific backgrounds, addressing one the identified challenges in building extensive mechanistic models as discussed above. Simcyp may combine the received input with its extensive libraries on demographics, developmental physiology, and the ontogeny of drug elimination pathways. As an indication of broad applicability, Simcyp can also be applied to variety of drug types, including small molecules, biologics, antibody-drug conjugates, generics, new modality drugs. The resulting model is automatically translated into a mathematical framework based on differential equations and implemented in a variety of programming languages to facilitate model sharing, validation, and regulatory submissions. The produced code enables tracking the behavior of drugs in relevant body tissues and organs. As a result, Simcyp links in vitro and in vivo ADME with pharmacokinetic/pharmacodynamic outcomes, providing the ability to explore clinical scenarios and support drug development directions. For example, Simcyp allows users to determine first-in-human dosing to answer translational questions. It also allows users to reduce trial size and complexity by aiding in determining clinical trial outcomes. Simcyp can quantitatively evaluate drug-drug interactions to predict variability in pharmacokinetic as a function of such factors as ethnicity and organ impairment. The predicted outcomes can allow users to provide dosing recommendations for different populations of patients, including pediatrics, geriatrics, ethnicities, and organ impairment.

### Illustrative Systems and Methods for Producing Mechanistic Models

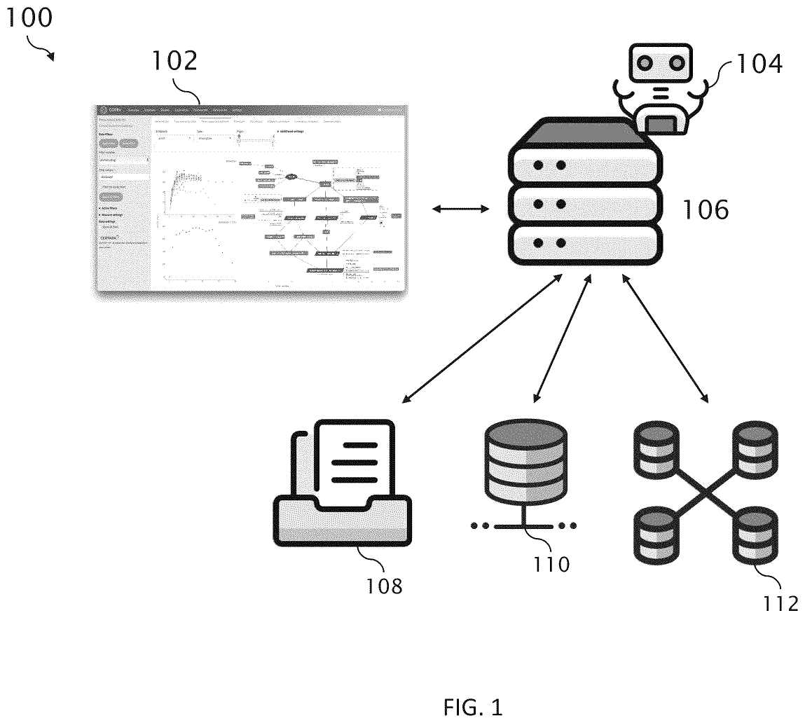

FIG. 1 illustrates an exemplary system 100 according to illustrative embodiments. The system 100 includes a mechanistic model graphical user interface (GUI) 102 that visualizes a mechanistic model, for example stored on a memory. The GUI 102 is connected to an interface architecture 106 that runs a machine-learning model 104 (e.g., a GPT). The interface architecture 106 can interact with a corpus of source data, including, for example, proprietary documents 108, proprietary databases 110, and/or public data 112 (e.g., a data mesh or data fabric). The GUI 102 may be used to receive prompts from a user from which the machine-learning model 104 can determine responsive information. The responsive information may be provided to the user in the GUI 102 (or, in some embodiments, in another GUI, for example provided on the same screen as GUI 102). A user of the GUI 102 may use the information to update the mechanistic model using the GUI 102.

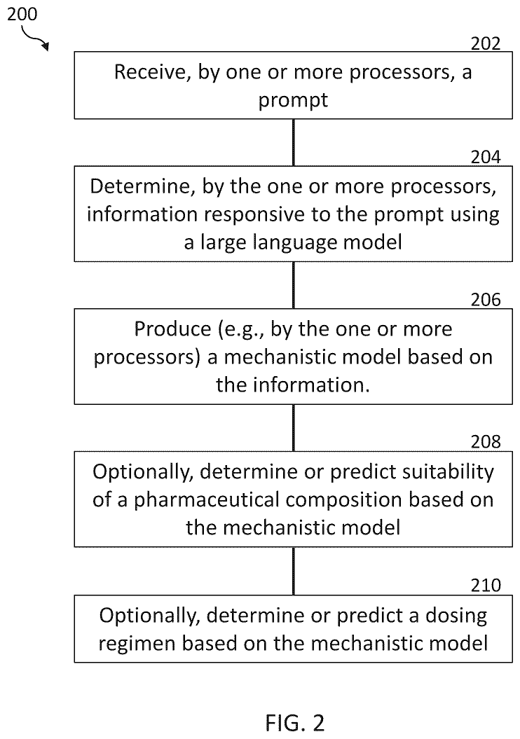

FIG. 2 is a flow diagram for exemplary method 200, which may be performed, for example, using system 100 illustrated in FIG. 1. In step 202, a prompt is received by one or more processors. In step 204, information responsive to the prompt is determined using a large language model. In step 206, a mechanistic model is produced based on the information (e.g., using the information). In certain embodiments, additional information is used as well. In certain embodiments “producing” a mechanistic model includes editing an existing mechanistic model. A human user may produce the mechanistic model in step 206, for example incorporating the information into the mechanistic model based on the human user's expertise in the field. In optional step 208, the mechanistic model is used to determine or predict suitability of a pharmaceutical composition. In optional step 210, a dosing regimen for a pharmaceutical composition is determined using the mechanistic model.

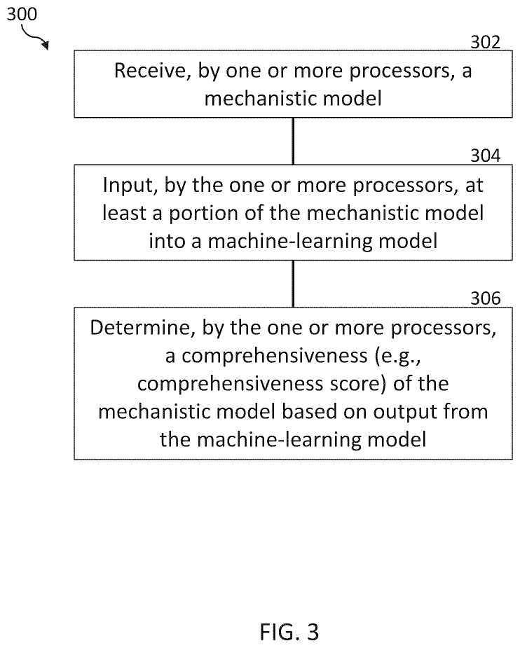

FIG. 3 is a flow diagram for exemplary method 300, which may be performed, for example, using system 100 illustrated in FIG. 1. In step 302, a mechanistic model is received by one or more processors. In step 304, at least a portion of a mechanistic model is input into a machine-learning model (e.g., in the form of one or more prompts). For example, one or more nodes and/or one or more associations in the mechanistic model may be input into the machine-learning model. In step 306, a comprehensiveness of the mechanistic model is determined based on output from the machine-learning model.

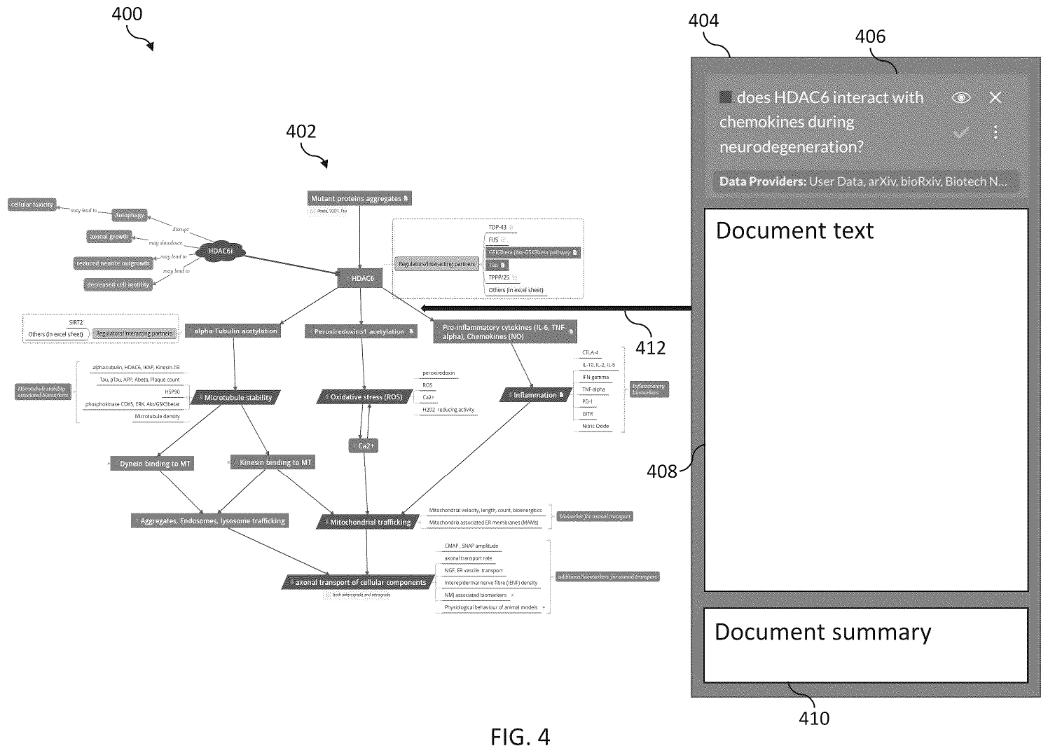

FIG. 4 is an illustration of graphical user interfaces (GUIs) 400 for producing mechanistic models for biological systems or processes using a large language model. The GUIs provide a graphical representation of a mechanistic model 402. The mechanistic model includes nodes and associations between the nodes (represented by arrows). A first GUI 404 can be used to display information responsive to a prompt related to a portion of the mechanistic model, in this case an association identified by arrow 412. The first GUI 404 includes a prompt widget 406 that displays a prompt and/or allows a user to enter a prompt (in this case “does HDAC6 interact with chemokines during neurodegeneration?”). Prompt widget 406 may also be used to select and/or display which corpus of source data is being used to determine information responsive to the prompt (in this case including User Data, arXiv, and bioRxiv). Information widget 408 displays information from a document responsive to the prompt shown in prompt widget 406 as determined by a machine-learning model. Document summary widget 410 displays a summary of the document that is the source of the information shown in information widget 408 (e.g., title, authors, etc.). A user may produce the mechanistic model 402 based on the information shown in the information widget 408. Data identifying the source of the information (e.g., shown in summary widget 410) may be incorporated into the mechanistic model 402 (e.g., as an annotation of a node or an association between nodes).

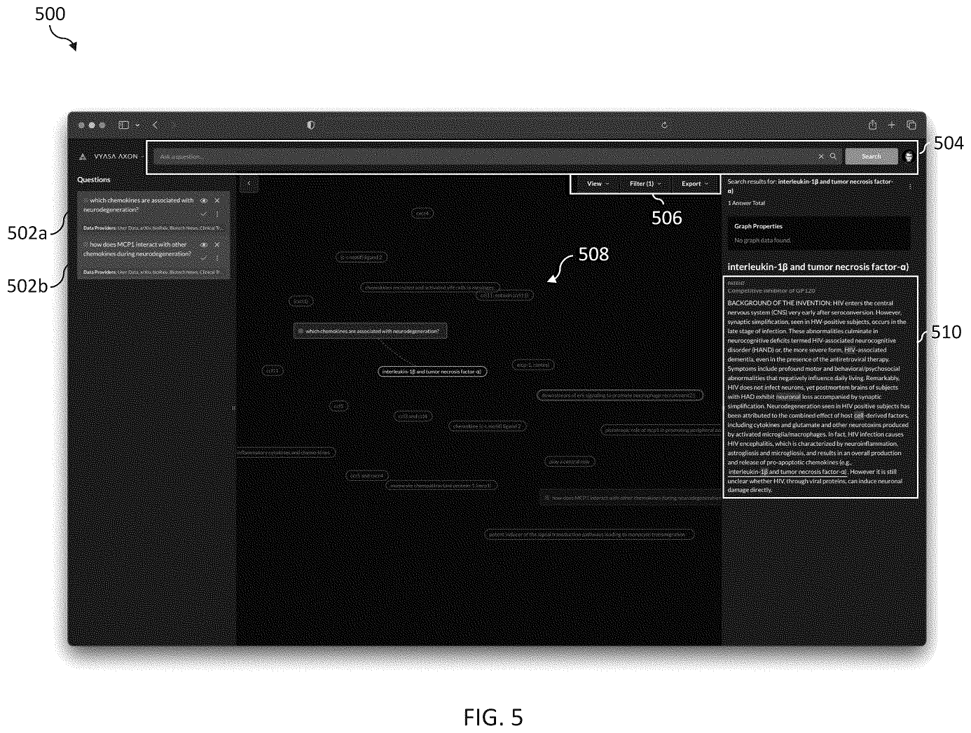

FIG. 5 is an illustration of a graphical user interface (GUI) 500 for using a large language model, according to illustrative embodiments of the present disclosure. GUI 500 includes prompt display and selection widgets 502a-b, prompt input widget 504, information display and processing settings widget 506, knowledge graph widget 508, and information widget 510. The prompt display and selection widgets 502a-b allow a user to select past prompts and view results responsive to the prompt. The prompt input widget 504 allows a user to input a new prompt. The information display and processing settings widget 506 allows a user to filter information display to the user, export the information, and other related settings. The knowledge graph widget 508 visualizes a knowledge graph for one or more selected prompts, where information responsive to the one or more selected prompts is displayed. Prompts are illustrated as central nodes that are connected to information responsive to the prompt. Information is shown in a summarized (e.g., condensed) format (e.g., by keyword) for simplicity of viewing. NLP may be used to determine which summary phrase or keyword is used to represent a large body of information in the knowledge graph 508. The information widget 510 shows more detailed information responsive to a prompt, for example including surrounding context and/or the information source. The information shown in information widget 510 is more detailed information that corresponds to the keyword or phrase selected in the knowledge graph widget 508. Selecting a different keyword or phrase in the knowledge graph widget 508 will change the information shown in the information widget 510. The information shown in the GUI 500 may be used to produce a mechanistic model, for example may be read and interpreted by a human user and then the user may produce the mechanistic model accordingly.

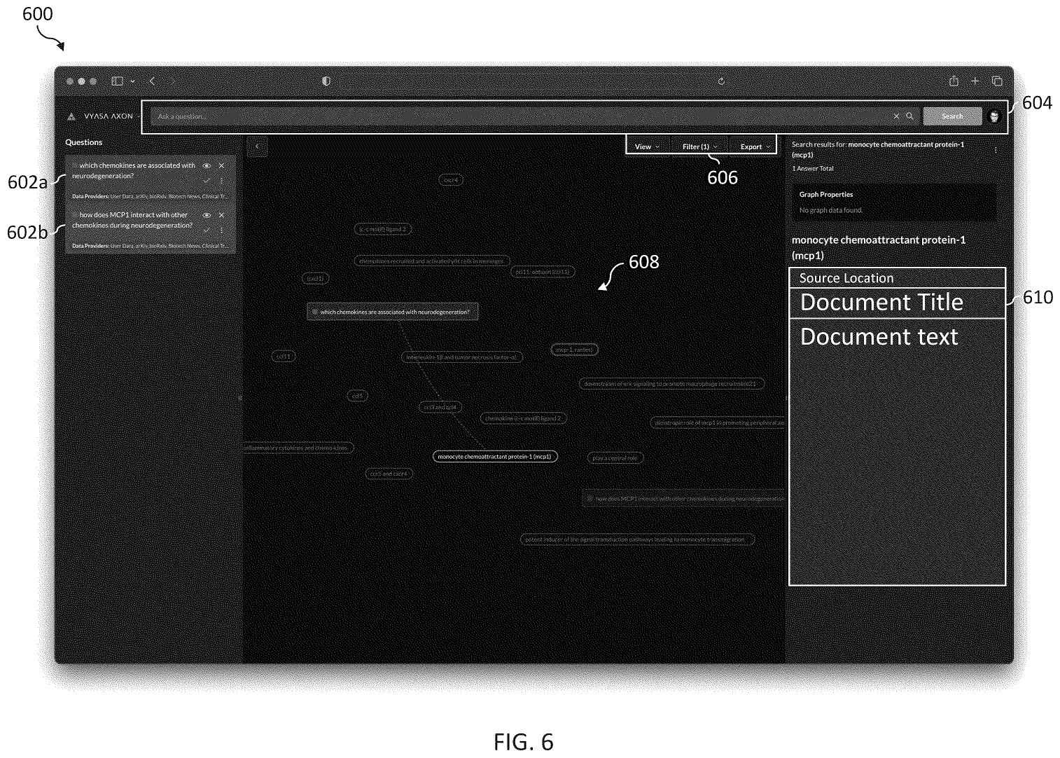

FIG. 6 is an illustration of a graphical user interface (GUI) 600 for using a large language model, according to illustrative embodiments of the present disclosure. GUI 600 includes prompt display and selection widgets 602a-b, prompt input widget 604, information display and processing settings widget 606, knowledge graph widget 608, and information widget 610. The prompt display and selection widgets 602a-b allow a user to select past prompts and view results responsive to the prompt. The prompt input widget 604 allows a user to input a new prompt. The information display and processing settings widget 606 allows a user to filter information display to the user, export the information, and other related settings. The knowledge graph widget 608 visualizes a knowledge graph for one or more selected prompts, where information responsive to the one or more selected prompts is displayed. Prompts are illustrated as central nodes that are connected to information responsive to the prompt. Information is shown in a summarized (e.g., condensed) format (e.g., by keyword) for simplicity of viewing. NLP may be used to determine which summary phrase or keyword is used to represent a large body of information in the knowledge graph 608. The information widget 610 shows more detailed information responsive to a prompt, in this case including a source location of the information, a document title for the information, and text from the document comprised in the information. The information shown in information widget 610 is more detailed information that corresponds to the keyword or phrase selected in the knowledge graph widget 608. Selecting a different keyword or phrase in the knowledge graph widget 608 will change the information shown in the information widget 610.

### Computer Software, Computer System, and Network Environment

Certain embodiments described herein make use of computer algorithms in the form of software instructions executed by a computer processor. In certain embodiments, the software instructions include a machine learning module, also referred to herein as artificial intelligence software. As used herein, a machine learning module refers to a computer implemented process (e.g., a software function) that implements one or more specific machine learning techniques, e.g., artificial neural networks (ANNs), e.g., convolutional neural networks (CNNs), e.g., recursive neural networks, e.g., recurrent neural networks such as long short-term memory (LSTM) or Bilateral long short-term memory (Bi-LSTM), random forest, decision trees, support vector machines, and the like, in order to determine, for a given input, one or more output values.

In certain embodiments, the methods and systems described herein make use of natural language processing (NLP) software, including but not limited to generative AI software (e.g., ChatGPT and/or proprietary generative AI software). In certain embodiments, this NLP software makes use of one or more large language models (LLMs). In certain embodiments, the one or more LLMs may be partially or wholly proprietary. In certain embodiments, the one or more LLMs may include one or more of the following known LLMs: BERT (Google) (or other transformer-based models), Falcon 40B, Galactica, GPT-3 (Generative Pre-trained Transformer, OpenAI), GPT-3.5 (OpenAI), GPT-4 (OpenAI), LaMDA (language model for dialogue applications, Google), Llama (large language model Meta AI) (Meta), Orca LLM (Microsoft), PaLM (Pathways Language Model), Phi-1 (Microsoft), StableLM (Stability AI), BLOOM (Hugging Face), ROBERTa (Meta), XLM-ROBERTa (Meta), NeMO LLM (Nvidia), XLNet (Google), Generate (Cohere), GLM-130B (Hugging Face), Claude (Anthropic). The one or more LLMs may include one or more autoregressive LLMs, autoencoding LLMs, encoder-decoder LLMs, bidirectional LLMs, Fine-tuned LLMs, and/or multimodal LLMs.

In certain embodiments, machine learning modules implementing machine learning techniques are trained, for example using datasets that include categories of data described herein (e.g., a corpus of source data). Such training may be used to determine various parameters of machine learning algorithms implemented by a machine learning module, such as weights associated with layers in neural networks. In certain embodiments, once a machine learning module is trained, e.g., to accomplish a specific task, such as determining information from a corpus of source data that is responsive to a prompt, values of determined parameters are fixed and the (e.g., unchanging, static) machine learning module is used to process new data (e.g., different from the training data) and accomplish its trained task without further updates to its parameters (e.g., the machine learning module does not receive feedback and/or updates). In certain embodiments, machine learning modules may receive feedback, e.g., based on user review of accuracy, and such feedback may be used as additional training data, to dynamically update the machine learning module. In certain embodiments, two or more machine learning modules may be combined and implemented as a single module and/or a single software application. In certain embodiments, two or more machine learning modules may also be implemented separately, e.g., as separate software applications. A machine learning module may be software and/or hardware. For example, a machine learning module may be implemented entirely as software, or certain functions of a ANN module may be carried out via specialized hardware (e.g., via an application specific integrated circuit (ASIC)).

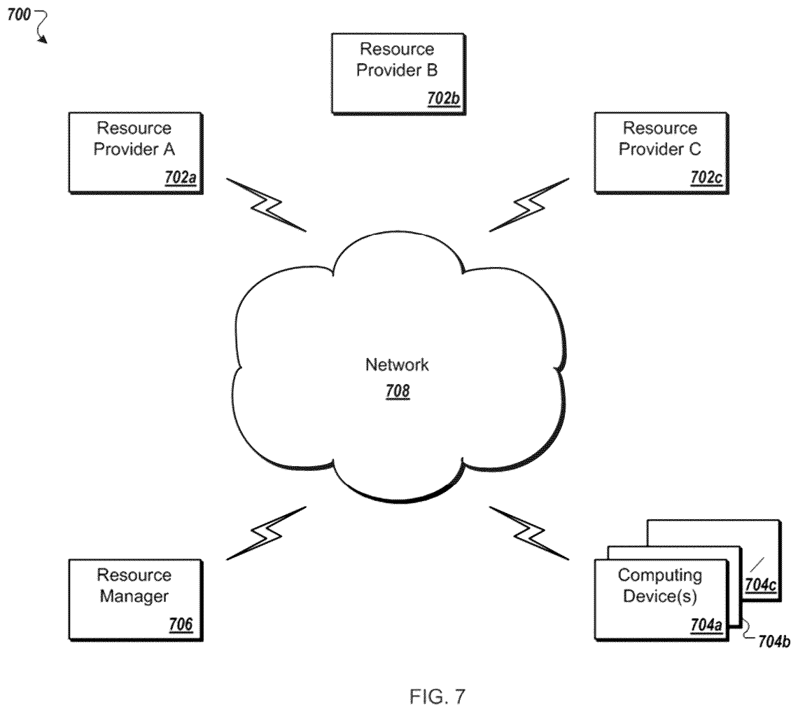

As shown in FIG. 7, an implementation of a network environment 700 for use in providing systems, methods, and architectures as described herein is shown and described. In brief overview, referring now to FIG. 7, a block diagram of an exemplary cloud computing environment 700 is shown and described. The cloud computing environment 700 may include one or more resource providers 702a, 702b, 702c (collectively, 702). Each resource provider 702 may include computing resources. In some implementations, computing resources may include any hardware and/or software used to process data. For example, computing resources may include hardware and/or software capable of executing algorithms, computer programs, and/or computer applications. In some implementations, exemplary computing resources may include application servers and/or databases with storage and retrieval capabilities. Each resource provider 702 may be connected to any other resource provider 702 in the cloud computing environment 700. In some implementations, the resource providers 702 may be connected over a computer network 708. Each resource provider 702 may be connected to one or more computing device 704a, 704b, 704c (collectively, 704), over the computer network 708.

The cloud computing environment 700 may include a resource manager 706. The resource manager 706 may be connected to the resource providers 702 and the computing devices 704 over the computer network 708. In some implementations, the resource manager 706 may facilitate the provision of computing resources by one or more resource providers 702 to one or more computing devices 704. The resource manager 706 may receive a request for a computing resource from a particular computing device 704. The resource manager 706 may identify one or more resource providers 702 capable of providing the computing resource requested by the computing device 704. The resource manager 706 may select a resource provider 702 to provide the computing resource. The resource manager 706 may facilitate a connection between the resource provider 702 and a particular computing device 704. In some implementations, the resource manager 706 may establish a connection between a particular resource provider 702 and a particular computing device 704. In some implementations, the resource manager 706 may redirect a particular computing device 704 to a particular resource provider 702 with the requested computing resource.

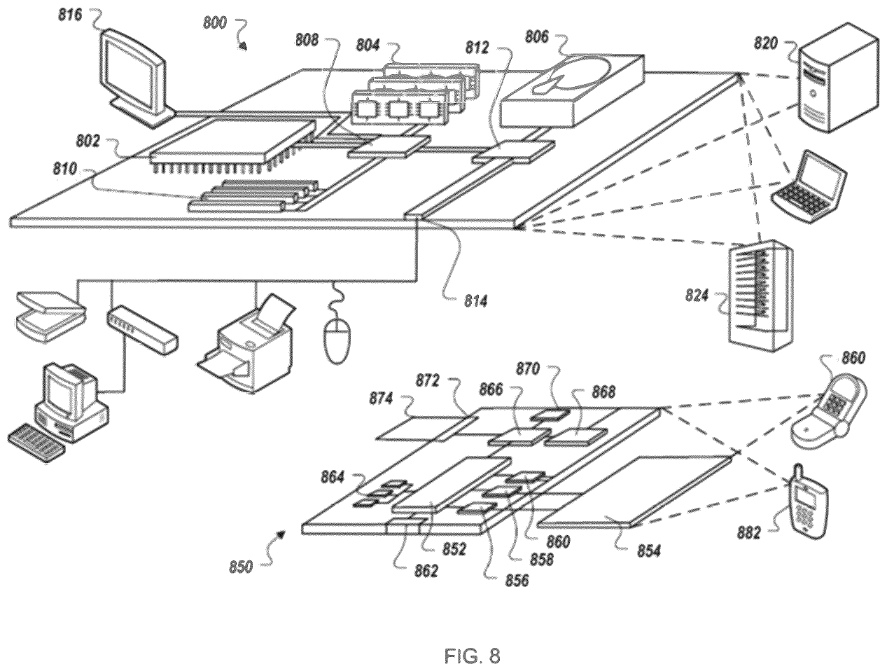

FIG. 8 shows an example of a computing device 800 and a mobile computing device 850 that can be used to implement the methods and systems described herein. The computing device 800 is intended to represent various forms of digital computers, such as laptops, desktops, workstations, personal digital assistants, servers, blade servers, mainframes, and other appropriate computers. The mobile computing device 850 is intended to represent various forms of mobile devices, such as personal digital assistants, cellular telephones, smart-phones, and other similar computing devices. The components shown here, their connections and relationships, and their functions, are meant to be examples only, and are not meant to be limiting.

The computing device 800 includes a processor 802, a memory 804, a storage device 806, a high-speed interface 808 connecting to the memory 804 and multiple high-speed expansion ports 810, and a low-speed interface 812 connecting to a low-speed expansion port 814 and the storage device 806. Each of the processor 802, the memory 804, the storage device 806, the high-speed interface 808, the high-speed expansion ports 810, and the low-speed interface 812, are interconnected using various busses, and may be mounted on a common motherboard or in other manners as appropriate. The processor 802 can process instructions for execution within the computing device 800, including instructions stored in the memory 804 or on the storage device 806 to display graphical information for a GUI on an external input/output device, such as a display 816 coupled to the high-speed interface 808. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system). Thus, as the term is used herein, where a plurality of functions are described as being performed by “a processor”, this encompasses embodiments wherein the plurality of functions are performed by any number of processors (one or more) of any number of computing devices (one or more). Furthermore, where a function is described as being performed by “a processor”, this encompasses embodiments wherein the function is performed by any number of processors (one or more) of any number of computing devices (one or more) (e.g., in a distributed computing system).

The memory 804 stores information within the computing device 800. In some implementations, the memory 804 is a volatile memory unit or units. In some implementations, the memory 804 is a non-volatile memory unit or units. The memory 804 may also be another form of computer-readable medium, such as a magnetic or optical disk.

The storage device 806 is capable of providing mass storage for the computing device 800. In some implementations, the storage device 806 may be or contain a computer-readable medium, such as a floppy disk device, a hard disk device, an optical disk device, or a tape device, a flash memory or other similar solid state memory device, or an array of devices, including devices in a storage area network or other configurations. Instructions can be stored in an information carrier. The instructions, when executed by one or more processing devices (for example, processor 802), perform one or more methods, such as those described above. The instructions can also be stored by one or more storage devices such as computer- or machine-readable mediums (for example, the memory 804, the storage device 806, or memory on the processor 802).

The high-speed interface 808 manages bandwidth-intensive operations for the computing device 800, while the low-speed interface 812 manages lower bandwidth-intensive operations. Such allocation of functions is an example only. In some implementations, the high-speed interface 808 is coupled to the memory 804, the display 816 (e.g., through a graphics processor or accelerator), and to the high-speed expansion ports 810, which may accept various expansion cards (not shown). In the implementation, the low-speed interface 812 is coupled to the storage device 806 and the low-speed expansion port 814. The low-speed expansion port 814, which may include various communication ports (e.g., USB, Bluetooth®, Ethernet, wireless Ethernet) may be coupled to one or more input/output devices, such as a keyboard, a pointing device, a scanner, or a networking device such as a switch or router, e.g., through a network adapter.

The computing device 800 may be implemented in a number of different forms, as shown in the figure. For example, it may be implemented as a standard server 820, or multiple times in a group of such servers. In addition, it may be implemented in a personal computer such as a laptop computer 822. It may also be implemented as part of a rack server system 824. Alternatively, components from the computing device 800 may be combined with other components in a mobile device (not shown), such as a mobile computing device 850. Each of such devices may contain one or more of the computing device 800 and the mobile computing device 850, and an entire system may be made up of multiple computing devices communicating with each other.

The mobile computing device 850 includes a processor 852, a memory 864, an input/output device such as a display 854, a communication interface 866, and a transceiver 868, among other components. The mobile computing device 850 may also be provided with a storage device, such as a micro-drive or other device, to provide additional storage. Each of the processor 852, the memory 864, the display 854, the communication interface 866, and the transceiver 868, are interconnected using various buses, and several of the components may be mounted on a common motherboard or in other manners as appropriate.

The processor 852 can execute instructions within the mobile computing device 850, including instructions stored in the memory 864. The processor 852 may be implemented as a chipset of chips that include separate and multiple analog and digital processors. The processor 852 may provide, for example, for coordination of the other components of the mobile computing device 850, such as control of user interfaces, applications run by the mobile computing device 850, and wireless communication by the mobile computing device 850.

The processor 852 may communicate with a user through a control interface 858 and a display interface 856 coupled to the display 854. The display 854 may be, for example, a TFT (Thin-Film-Transistor Liquid Crystal Display) display or an OLED (Organic Light Emitting Diode) display, or other appropriate display technology. The display interface 856 may comprise appropriate circuitry for driving the display 854 to present graphical and other information to a user. The control interface 858 may receive commands from a user and convert them for submission to the processor 852. In addition, an external interface 862 may provide communication with the processor 852, so as to enable near area communication of the mobile computing device 850 with other devices. The external interface 862 may provide, for example, for wired communication in some implementations, or for wireless communication in other implementations, and multiple interfaces may also be used.

The memory 864 stores information within the mobile computing device 850. The memory 864 can be implemented as one or more of a computer-readable medium or media, a volatile memory unit or units, or a non-volatile memory unit or units. An expansion memory 874 may also be provided and connected to the mobile computing device 850 through an expansion interface 872, which may include, for example, a SIMM (Single In Line Memory Module) card interface. The expansion memory 874 may provide extra storage space for the mobile computing device 850, or may also store applications or other information for the mobile computing device 850. Specifically, the expansion memory 874 may include instructions to carry out or supplement the processes described above, and may include secure information also. Thus, for example, the expansion memory 874 may be provide as a security module for the mobile computing device 850, and may be programmed with instructions that permit secure use of the mobile computing device 850. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. In some implementations, instructions are stored in an information carrier. The instructions, when executed by one or more processing devices (for example, processor 852), perform one or more methods, such as those described above. The instructions can also be stored by one or more storage devices, such as one or more computer- or machine-readable mediums (for example, the memory 864, the expansion memory 874, or memory on the processor 852). In some implementations, the instructions can be received in a propagated signal, for example, over the transceiver 868 or the external interface 862.

The mobile computing device 850 may communicate wirelessly through the communication interface 866, which may include digital signal processing circuitry where necessary. The communication interface 866 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 868 using a radio-frequency. In addition, short-range communication may occur, such as using a Bluetooth®, Wi-Fi™, or other such transceiver (not shown). In addition, a GPS (Global Positioning System) receiver module 870 may provide additional navigation- and location-related wireless data to the mobile computing device 850, which may be used as appropriate by applications running on the mobile computing device 850.

The mobile computing device 850 may also communicate audibly using an audio codec 860, which may receive spoken information from a user and convert it to usable digital information. The audio codec 860 may likewise generate audible sound for a user, such as through a speaker, e.g., in a handset of the mobile computing device 850. Such sound may include sound from voice telephone calls, may include recorded sound (e.g., voice messages, music files, etc.) and may also include sound generated by applications operating on the mobile computing device 850.

The mobile computing device 850 may be implemented in a number of different forms, as shown in the figure. For example, it may be implemented as a cellular telephone 880. It may also be implemented as part of a smart-phone 882, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

In some implementations, certain modules described herein can be separated, combined or incorporated into single or combined modules. Any modules depicted in the figures are not intended to limit the systems described herein to the software architectures shown therein.

Elements of different implementations described herein may be combined to form other implementations not specifically set forth above. Elements may be left out of the processes, computer programs, databases, etc. described herein without adversely affecting their operation. In addition, the logic flows depicted in the figures do not require the particular order shown, or sequential order, to achieve desirable results. Various separate elements may be combined into one or more individual elements to perform the functions described herein.

Headers have been provided in the foregoing for the convenience of the reader and are not intended to be limiting with respect to any subject matter.

It is contemplated that systems, devices, methods, and processes of the disclosure encompass variations and adaptations developed using information from the embodiments described herein. Adaptation and/or modification of the systems, devices, methods, and processes described herein may be performed by those of ordinary skill in the relevant art.

Throughout the description, where articles, devices, and systems are described as having, including, or comprising specific components, or where processes and methods are described as having, including, or comprising specific steps, it is contemplated that, additionally, there are articles, devices, and systems according to certain embodiments of the present disclosure that consist essentially of, or consist of, the recited components, and that there are processes and methods according to certain embodiments of the present disclosure that consist essentially of, or consist of, the recited steps.

It should be understood that the order of steps or order for performing certain action is immaterial so long as operability is not lost. Moreover, two or more steps or actions may be conducted simultaneously.

Certain embodiments of the present disclosure were described above. It is, however, expressly noted that the present disclosure is not limited to those embodiments, but rather the intention is that additions and modifications to what was expressly described in the present disclosure are also included within the scope of the disclosure. Moreover, it is to be understood that the features of the various embodiments described in the present disclosure were not mutually exclusive and can exist in various combinations and permutations, even if such combinations or permutations were not made express, without departing from the spirit and scope of the disclosure. The disclosure has been described in detail with particular reference to certain embodiments thereof, but it will be understood that variations and modifications can be effected within the spirit and scope of the claimed invention.

1. A method of producing a mechanistic model using information derived using artificial intelligence (AI), the method comprising:
receiving, by one or more processors of one or more computing devices, a prompt;
determining, by the one or more processors, information responsive to the prompt, wherein determining the information comprises inputting the prompt into a large language model (LLM) and outputting the information from the LLM; and
producing a mechanistic model based on the information.

2. The method of claim 1, wherein the LLM processes the prompt using a defined corpus of source data such that the information is derived from the corpus of source data.

3. The method of claim 2, wherein the source data comprise documents, data identified by IDs, data identified by hyperlinks, or a combination thereof.

4. The method of claim 2, wherein the source data comprise public data and the information is derived from the public data.

5-9. (canceled)

10. The method of claim 1, comprising associating a portion of the mechanistic model that has been produced with a source of the information.

11. The method of claim 1, wherein the information is output from the LLM with a source of the information already associated.

12. The method of claim 1, wherein producing the mechanistic model comprises incorporating a source of the information in the mechanistic model such that the source of the information is determinable from the mechanistic model.

13-17. (canceled)

18. The method of claim 1, comprising determining, by the one or more processors, using the LLM, a plurality of answers to the prompt and a quality metric for each of the answers, wherein the information comprises one or more of the plurality of answers each having a respective quality metric that is higher than a respective quality of one or more other of the plurality of answers.

19. The method of claim 18, comprising providing, by the one or more processors, the information and the one or more other of the plurality of answers to a user in a graphical user interface (GUI) in a format that is based on quality.

20. The method of claim 18, comprising providing, by the one or more processors, the information in a graphical user interface (GUI) in a format that is based on quality and not providing the one or more other of the plurality of answers to a user in the GUI.

21-23. (canceled)

24. The method of claim 1, comprising continuously updating the mechanistic model using the LLM.

25-34. (canceled)

35. The method of claim 1, wherein the LLM is a base model.

36. The method of claim 1, wherein the LLM is a fine-tuned model.

37. The method of claim 1, wherein the LLM is a commercially available model.

38. The method of claim 1, wherein the LLM is a purpose-built model.

39. The method of claim 1, wherein the LLM is a generative pre-trained transformer (GPT).

40. The method of claim 1, wherein the LLM is a foundation model.

41. (canceled)

42. The method of claim 1, wherein the mechanistic model comprises a quantitative systems pharmacology (QSP) model or physiologically based pharmacokinetics (PBPK) model.

43. The method of claim 1, wherein the mechanistic model is a model for a biological system and/or a biological process.

44-53. (canceled)

54. A system comprising the one or more processors and one or more non-transitory computer readable media, wherein the one or more non-transitory computer readable media have instructions stored thereon that, when executed by the one or more processors, cause the one or more processors to perform operations comprising the method of claim 1.

55-82. (canceled)
